(19) United States
(12) Patent Application Publication
(10) Pub. No.: US 2026/0109913 A1
(43) Pub. Date: Apr. 23, 2026

(54) A PROCESS FOR REDUCING DIGLYCERIDES

(71) Applicant: Novozymes A/S, Bagsvaerd (DK)

(72) Inventors: Rasmus Boeg HANSEN, Frederiksberg (DK); Hon Seng YEE, Kuala Lumpur (MY); Kim BORCH, Birkeroed (DK)

(73) Assignee: Novozymes A/S, Bagsvaerd (DK)

(21) Appl. No.: 19/120,501

(22) PCT Filed: Oct. 13, 2023
(86) PCT No.: PCT/EP2023/078440

§ 371 Date: Apr. 11, 2025

(30) Foreign Application Priority Data
Oct. 14, 2022 (MY) PI2022005761

(51) Int. Cl. C11B 3/00; C11C 1/04; C11C 1/08; C12N 9/20; C12P 7/6418

(52) U.S. Cl. CPC C11B 3/003; C11C 1/04; C11C 1/08; C12N 9/20; C12P 7/6418; C12Y 301/01003

(57) ABSTRACT

A process for reducing and/or removing diglyceride content without substantial interesterification of triglycerides in an oil comprising steps of providing an oil or fat; and hydrolysis of mono- and diglycerides in said oil with water in the presence of a lipase having at least 80% sequence identity to SEQ ID NO: 1.

### FIELD OF INVENTION

The present invention relates to a process for the enzymatic removal and/or reduction of diglyceride from an oil.

### BACKGROUND OF THE INVENTION

Oils and fats consist of complex mixtures of triacylglycerols (TAGs), diacylglycerols (DAGs), monoacylglycerols (MAGs), free fatty acids and other minor components. The crystallisation of these mixtures depends on the characteristics of the TAGs (combination of fatty acids, their chain length, their degree of unsaturation, and the like) and the interaction of these TAGs with each other. Regarding the presence of DAGs, previous studies have shown that they have significant effect on the physical properties of oils and fats. These vary from rate of crystallisation, polymorphism changes, melting point, crystal size and habits (Siew, 2001).

In some oils that are extracted from oilseeds, the effect of DAG is less pronounced, as DAG is often only present in small quantities. But in e.g. palm oil, rice bran, shea butter and olive oil, there is typically a high amount of DAG after extraction, and the quality of these oils suffers if DAG are present therein.

The presence of these diglycerides in the main product (=triglycerides) is unfavourable as the diglycerides have an adverse effect on the product properties of the triglycerides. Diglycerides are also reactive during parts of the refining process, especially during deodorization, and tend to form food-safety-regulated byproducts at certain conditions, e.g. glycidyl esters.

A number of processes coping with this problem are disclosed in literature. These are focussed on the removal of diglycerides from mixtures with triglycerides, wherein an enzymic conversion of diglycerides is carried out by using an enzyme specific for the hydrolysis of diglycerides into glycerol and free fatty acids.

In JP 62/51997, e.g., a method for the improvement of fats is disclosed, wherein a mixture containing at least 70 weight percent of triglycerides and not less than 2 weight percent of diglycerides is contacted with an enzyme specific for partial glycerides in the presence of a small amount of water.

A similar process is disclosed in JP 62/61590. The hydrolysis of the partial glycerides is now followed by an esterification process using a 1,3-specific enzyme.

In JP 62/287 a similar process is disclosed by which the hydrolysis of monoglycerides and/or diglycerides is carried out by using the lipase produced by Penicillium cyclopium ATCC 34613.

The prior art thus teaches ways to reduce or remove the content of diglyceride in palm oil and other edible oils by enzymatic reactions. These processes rely on the hydrolysis of diglyceride with a specific diglyceride hydrolysing lipase during formation of free fatty acids and glycerol. The free fatty acids can then be removed by means of different processes like vacuum distillation, saponification or fractionation.

Using a specific diglyceride hydrolysing enzyme leads to formation of free fatty acids. These free fatty acids often must be removed from the oil. But in the current market situation, where fatty acid distillate is relatively valuable for various reasons, the combination of improvement in TAG oil quality at the cost of additional production of FFA (often distillate) can bring a combined improvement in the economics. An added advantage of reducing MAG and DAG, is a significant quality increase of the distillate relative to standard quality can even be expected, because hydrolysis of MAG and DAG leads to increased FFA concentration in the distillate. This means such distillates will need less extensive treatments, saving energy consumption. To bring such an economical benefit a sufficiently cheap lipase is required, and to our knowledge there is no such lipase available today.

The present invention provides a solution of using a lipase to overcome the problems with high diglyceride content in palm oil and other vegetable oils and derivatives all the way through the supply chain. The lipase is sufficiently easy to produce to enable employment at producers today at a sufficiently low cost to enable economically feasible production of oils with the above-mentioned benefits of low diglycerides, not least on health.

### SUMMARY OF THE INVENTION

A process for reducing and/or removing diglyceride content without substantial interesterification of triglycerides in an oil comprising steps of: providing an oil or fat; and hydrolysis of mono- and diglycerides in said oil with water in the presence of a lipase having at least 80% sequence identity to SEQ ID NO: 1.

A general objective of the present invention is to provide a process for the enzymatic removal and/or reduction of diglyceride from an oil which allows for a profitable competitive large-scale process.

This objective is achieved by means of the features of each one of the independent claims. Advantageous further embodiments are defined in the sub-claims.

These and still other objectives and advantages of the present invention will be apparent from the description which follows. In the detailed description below, preferred embodiments of the invention will be described in reference to the accompanying drawings. These embodiments do not represent the full scope of the invention. Rather the invention may be employed in other embodiments. Reference should therefore be made to the claims herein for interpreting the breadth of the invention.

### Definitions

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a step” includes reference to one or more of such steps.

As used herein, “substantial” when used in reference to a quantity or amount of a material, or a specific characteristic thereof, refers to an amount that is sufficient to provide an effect that the material or characteristic was intended to provide. The exact degree of deviation allowable may in some cases depend on the specific context. Similarly, “substantially free of” or the like refers to the lack of an identified element or agent in a process. Particularly, elements that are identified as being “substantially free of” are either completely absent from the process or are included only in amounts which are small enough so as to have no deleterious effect on the process.

Reference to “about” a value or parameter herein includes embodiments that are directed to that value or parameter per se. For example, description referring to “about X” includes the embodiment “X”. When used in combination with measured values, “about” includes a range that encompasses at least the uncertainty associated with the method of measuring the particular value and can include a range of plus or minus two standard deviations around the stated value.

Likewise, reference to a gene or polypeptide that is “derived from” another gene or polypeptide X, includes the gene or polypeptide X.

It is understood that the embodiments described herein include “consisting” and/or “consisting essentially of” embodiments. As used herein, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of about 1 percent to about 20 percent should be interpreted to include not only the explicitly recited concentration limits of 1 percent to about 20 percent, but also to include individual concentrations such as 2 percent, 3 percent, 4 percent, and sub-ranges such as 5 percent to 15 percent, 10 percent to 20 percent, etc.

The term “lipid” refers to phospholipids and their derivatives, triglycerides and derivatives, sterols, stanols, cholesterol, sphingolipids, ceramides, fatty acids, fatty alcohols, glycolipids, proteolipids, lipopolysaccharides, ether-lipids, polar and non-polar lipids and derivatives thereof.

The term “esterification” as used herein, refers to a reaction for combining an organic acid such as a fatty acid with any alcohol or polyol such as a glycerol.

The term “hydrolysis” as used herein, refers to the reaction of water with an ester to produce an acid and an alcohol.

The term “interesterification” as used herein, refers to the reaction of a first ester with a second ester leading to a mix up between the acyl and the alcohol moieties.

The terms “alkyl” or “alkyl group” is to be construed according to its broadest meaning, to describe a univalent aliphatic compound comprising hydrocarbons.

The terms “glycerol derivatives” and “glycerides” are interchangeably used herein to describe esters, ethers and other derivatives of glycerol in which at least one of the hydrogens, of any of the hydroxyl group attached to the C1, C2 or C3 carbons, is substituted. Examples of glycerol derivatives are: tristearoylglycerol (or tri-Ostearoyl glycerol or glycerol tristearate, or glyceryl tristearate); I,3-benzylideneglycerol (or 1,3-0-benzylideneglycerol); and glycerol 2-phosphate (or 2-phosphoglycerol) among others. If the substitution is on a carbon atom, rather than on the oxygen of the hydroxyl group than the compound may be considered as a derivative of glycerol (e.g., 1,2,3-nonadecanetriol for C16H33CHOH—CHOH—CH2OH, which may be also considered as 1-C-hexadecyl glycerol). The term “glycerol” as used herein is intended to encompass glycerol derivatives including glycerol.

The terms mono-, di- and tri-glycerol/glycerides, mono-, di- and tri-acylglycerol/acylglycerides, MG/DG/TG and MAG/DAG/TAG are used herein interchangeably and all refer to fatty acid based glycerides.

Lipase: The terms “lipase”, “lipase enzyme”, “lipolytic enzyme”, “lipid esterase”, “lipolytic polypeptide”, and “lipolytic protein” refers to an enzyme in class EC3.1.1 as defined by Enzyme Nomenclature. It may have lipase activity (triacylglycerol lipase, EC3.1.1.3), cutinase activity (EC3.1.1.74), sterol esterase activity (EC3.1.1.13) and/or wax-ester hydrolase activity (EC3.1.1.50).

The term “parent” or “parent lipase” means a lipase to which an alteration is made to produce the enzyme variants. The parent lipase may be a naturally occurring (wild-type) polypeptide but may also be a variant and/or fragment thereof.

The relatedness between two amino acid sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

Suitable substrates in accordance with the present invention are a broad variety of vegetable oils and fats; rapeseed and soybean oils are most commonly used, though other crops such as mustard, sunflower, canola, coconut, hemp, palm oil and even algae show promise. The substrate can be of crude quality or further processed (refined, bleached and deodorized). Also, animal fats including tallow, lard, poultry, marine oil as well as waste vegetable and animal fats and oil, commonly known as yellow and brown grease can be used. The suitable fats and oils may be pure triglyceride or a mixture of triglyceride and free fatty acids, commonly seen in waste vegetable oil and animal fats. The substrate may also be obtained from vegetable oil deodorizer distillates. The type of fatty acids in the substrate comprises those naturally occurring as glycerides in vegetable and animal fats and oils. These include oleic acid, linoleic acid, linolenic acid, palmitic acid, stearic acid, and lauric acid to name a few. Minor constituents in crude vegetable oils are typically phospholipids, free fatty acids and partial glycerides i.e., mono- and diglycerides.

The term “fatty acid feedstock” or “oils and/or fats” or “vegetable oil feedstock” is defined herein as a substrate comprising fatty acid derivatives. The substrate may comprise fatty acid alkyl esters, triglyceride, diglyceride, monoglyceride, free fatty acid or any combination thereof. Any oils and fats of vegetable or animal origin comprising fatty acids may be used as substrate for producing fatty acid alkyl esters in the process of the invention. Also, fatty acid feedstock consisting substantially of fatty acid alkyl esters is suitable as feedstock (biodiesel feedstock) for the present invention.

The fatty acid feedstock may be oil selected from the group consisting of: microbial oil, algae oil, canola oil, coconut oil, castor oil, coconut oil (copra oil), corn oil, cottonseed oil, flax oil, fish oil, grape seed oil, hemp oil, jatropha oil, jojoba oil, mustard oil, canola oil, palm oil, distillers' corn oil, palm stearin, palm olein, palm kernel oil, peanut oil, rapeseed oil, rice bran oil, safflower oil, soybean oil, sunflower oil, tall oil, and oil from halophytes, pennycress oil, camelina oil, jojoba oil, coriander seed oil, meadowfoam oil, seashore mallow oil, or any combination thereof.

The fatty acid feedstock may be fat selected from the group consisting of animal fat, including tallow from pigs, beef and sheep, lard, chicken fat, fish oil, or any combination thereof.

The fatty acid feedstock may be crude, refined, bleached, deodorized, degummed, or any combination thereof.

The terms Free fatty acid (FFA) is a carboxylic acid with a long carbon chain. Most naturally occurring fatty acids have an unbranched chain of an even number of carbon atoms, from 4 to 24. Free fatty acids are usually derived from fats (triglycerides (TAG), diglycerides (DAG), monoglyceride (MAG)), phospholipids or lyso-phospholipids. Triglycerides are formed by combining glycerol with three fatty acid molecules. The hydroxyl (HO—) group of glycerol and the carboxyl (—COOH) group of the fatty acid join to form an ester. The glycerol molecule has three hydroxyl (HO—) groups. Each fatty acid has a carboxyl group (—COOH). Diglycerides are formed by combining glycerol with two fatty acid molecules. Monoglycerides are formed by combining glycerol with one fatty acid molecule.

### DETAILED DESCRIPTION OF THE INVENTION

The present invention relates to process for reducing and/or removing diglyceride content without substantial interesterification of triglycerides in oil.

In one aspect, the present invention relates to a process for reducing and/or removing diglycerides without substantial interesterification of triglycerides in oil comprising steps of: providing an oil or fat and hydrolysis of mono- and diglycerides in said oil with water in the presence of a lipase having at least 80% sequence identity to SEQ ID NO: 1.

In one aspect, the lipolytic enzyme or a lipase is applied in the process of the present invention is selected from lipases, phospholipases, cutinases, acyltransferases or a mixture of one and more of lipase, phospholipase, cutinase and acyltransferase. The lipolytic enzyme or the lipase is selected from the enzymes in EC 3.1.1, EC 3.1.4, and EC 2.3.

A suitable lipolytic enzyme may be a polypeptide having lipase activity, e.g., one selected from the Candida antarctica lipase A (CALA) as disclosed in WO 88/02775, the C. antarctica lipase B (CALB) as disclosed in WO 88/02775 and shown in SEQ ID NO:1 of WO2008065060 the Thermomyces lanuginosus (previously Humicola lanuginosus) lipase disclosed in EP 258 068), the Thermomyces lanuginosus variants disclosed in WO 2000/60063 or WO 1995/22615, in particular the lipase shown in positions 1-269 of SEQ ID NO: 2 of WO 95/22615, the Hyphozyma sp. lipase (WO 98/018912), and the Rhizomucor miehei lipase (SEQ ID NO:5 in WO 2004/099400), a lipase from P. alcaligenes or P. pseudoalcaligenes (EP 218 272), P. cepacia (EP 331 376), P. glumae, P. stutzeri (GB 1,372,034), P. fluorescens, Pseudomonas sp. strain SD 705 (WO 95/06720 and WO 96/27002), P. wisconsinensis (WO 96/12012); a Bacillus lipase, e.g., from B. subtilis (Dartois et al. (1993), Biochemica et Biophysica Acta, 1131, 253-360), B. stearothermophilus or G. stearothermophilus (JP 64/744992) or B. pumilus (WO 91/16422). Also preferred is a lipase from any of the following organisms: Fusarium oxysporum, Absidia reflexa, Absidia corymbefera, Rhizomucor miehei, Rhizopus delemar (oryzae), Aspergillus niger, Aspergillus tubingensis, Fusarium heterosporum, Aspergillus oryzae, Penicilium camembertii, Aspergillus foetidus, and Thermomyces lanuginosus, such as a lipase selected from any of SEQ ID NOs: 1 to 15 in WO 2004/099400.

### Lipase Activity:

In the context of the present invention, the lipase activity may be determined as lipase units (LU), using tributyrate as substrate. The method is based on the hydrolysis of tributyrin by the enzyme, and the alkali consumption to keep pH constant during hydrolysis is registered as a function of time

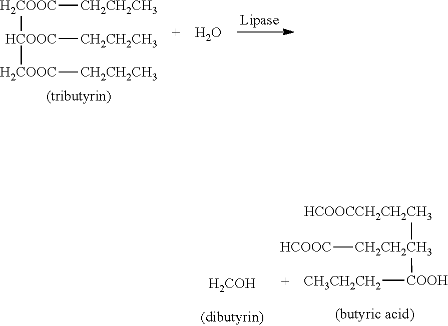

One lipase unit (LU) may be defined as the amount of enzyme which, under standard conditions (i.e. at 30° C.; pH 7.0; with 0.1% (w/v) Gum Arabic as emulsifier and 0.16 M tributyrine as substrate) liberates 1 micromol titrable butyric acid per minute.

Alternatively, lipolytic activity may be determined as Long Chain Lipase Units (LCLU) using substrate pNP-Palmitate (C: 16) when incubated at pH 8.0, 30° C., the lipase hydrolyzes the ester bond and releases pNP, which is yellow and can be detected at 405 nm.

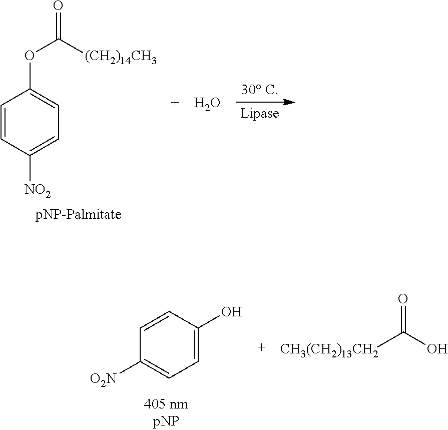

The term “selective” as used herein means that in an edible oil environment, the lipase utilizes diglycerides (DAGs), as a substrate preferentially to triacylglycerides (TAGs). Thus, diglycerides can be removed and/or reduced from the edible oil whilst leaving the amount of triglyceride in the oil unchanged (or substantially unchanged). The amount of monoglycerides in the oil is also substantially hydrolyzed during the treatment especially when sufficient amounts of water are available.

In an embodiment of the present invention, the lipase is a polypeptide having at least 80% sequence identity to SEQ ID NO:1.

In another embodiment, the lipase is a polypeptide having at least at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:1.

In a preferred embodiment of the present invention, the lipase is a polypeptide comprising, consisting essentially of, or consisting of SEQ ID NO: 1.

In an embodiment of the present invention, the lipase comprises or consists of the amino acid sequence shown in SEQ ID NO 1.

In an embodiment of the present invention, the oil is derived from one or more of algae oil, canola oil, coconut oil, castor oil, coconut oil, copra oil, corn oil, distiller's corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, shea oil, tall oil, oil from halophytes, and/or animal fat, including tallow from pigs, beef and sheep, lard, chicken fat, fish oil, palm oil free fatty acid distillate, soy oil free fatty acid distillate, soap stock fatty acid material, yellow grease, used cooking oil, palm oil mill effluent and brown grease or any combination thereof.

In an embodiment of the present invention, the process is performed at temperatures in the range of 10-100° C., preferably 20-90° C.

In an embodiment of the present invention, the hydrolysis which comprises reacting free fatty acids and/or a fatty acid in oil with water in presence of lipase until at least 30% (w/w) such as more that 50% (w/w) or such as at least 70% (w/w) of the fatty acid acyl groups of the DAG in said oil have been converted to free fatty acids.

In an embodiment of the present invention, the total amount of said lipase added during hydrolysis is within the range 0.1-50000 mg enzyme protein (EP)/kg of oil. Preferably 0.1-200 mg enzyme protein (EP)/kg of oil in cases where a liquid enzyme formulation is used, and preferably 500-50000 mg enzyme protein (EP)/kg of oil in cases where an immobilized enzyme formulation is used.

In an embodiment of the present invention, the lipase is preferably employed as a liquid product, an immobilized product or as a dry powder.

In an embodiment of the present invention, the total reaction time of the process is at least 15 minutes.

In an embodiment of the present invention, the total reaction time of the process is up to 48 hours.

In an embodiment of the present invention, the amount of water added during the hydrolysis is between 0.01 and 100% (w/w) of oil.

In an embodiment of the present invention, the pH is optionally adjusted during or prior to hydrolysis to optimize the reaction.

In an embodiment of the present invention, the pH during hydrolysis is between 3.0-7.0.

In an embodiment of the present invention, the pH is adjusted using citric acid, phosphoric acid, sodium hydroxide and/or potassium hydroxide.

In an embodiment of the present invention, the process is performed in a batch, semi-continuous or continuous mode.

In another embodiment of the present invention, the process runs in a number of sequential reaction steps such as 2-10 reactors in series, preferably 2-5 reactors in series.

In another embodiment of the present invention, the process is performed in a countercurrent, optionally compartmentalized, reactor.

In another embodiment of the present invention, the lipase is used in immobilized formulation using e.g. a column or bed.

In another embodiment of the present invention, the process further comprises addition of one or more additional lipase and/or a phospholipase during hydrolysis.

In an embodiment of the present invention, the amount of triglyceride in the oil is unchanged (or substantially unchanged) after treatment with lipase.

In another embodiment of the present invention, the feedstock oil or fat is the feedstock of a degumming process. Optionally this feedstock is treated according to the invention prior to degumming. Optionally this feedstock is treated according to the invention after degumming. Optionally this feedstock is treated according to the invention in combination with degumming. Such degumming can be e.g. existing plants conducting water degumming, acid degumming, enzymatic degumming, but is not limited to those.

In another embodiment of the present invention, the feedstock oil or fat is previously refined and/or bleached and the invention is utilized as a pretreatment prior to deodorization to improve the quality of the deodorized product by reducing production of undesired byproducts such as 3MCPD and glycidol esters during deodorization.

In another embodiment of the present invention, the feedstock oil or fat is intended for fractionation and/or winterization, and employment of the present invention is used to improve e.g. the yield of the desired fractions.

In another embodiment, the process of hydrolysis of diglycerides occurs sequentially or simultaneously degumming process.

In another embodiment, the process of hydrolysis of diglycerides occurs in the ‘miscella’ mixture of extracted oils prior to evaporation. This mixture is the mixture of oil and organic solvent leaving the main oil extraction step. The miscella mixture was then separated into product crude oil and organic solvent for reuse. This mixture mainly comprises of oil and organic solvent. Preferably the solvent is acetone, hexane or heptane. Most preferably the organic solvent is hexane. The method disclosed in this document can thus be employed on the miscella mixture by addition of enzyme and water, followed by a separation step, after which the treated mixture of oil and hexane can pass through the usual process of evaporation. This yields a crude oil of improved quality by reducing DAG and forming FFA even before entering the refinery and is advantageous for especially crushing plants that wish to offer a crude product of improved quality to external refiners.

The invention is further described in the following paragraphs.

Paragraph 1. A process for reducing and/or removing diglyceride content without substantial interesterification of triglycerides in an oil comprising steps of:
a. providing an oil or fat; and
b. hydrolysis of diglycerides in said oil with water in the presence of a lipase having at least 80% sequence identity to SEQ ID NO: 1.
   Paragraph 2. The process according to paragraph 1, further comprises the step of separation of light and heavy phase after hydrolysis.
   Paragraph 3. The process according to paragraph 2, wherein the light phase comprises the oil with reduced diglyceride and increased FFA.
   Paragraph 4. The process according to paragraph 2, wherein the heavy phase comprises water, lipase, glycerol.
   Paragraph 5. The process according to paragraphs 2-4, wherein the heavy phase is partially or fully recycled into hydrolysis step.
   Paragraph 6. The process according to paragraphs 2-4, wherein free fatty acid is separated from the oil present in the light phase.
   Paragraph 7. The process according to paragraph 1, wherein less than 10%, preferably less than 5%, more preferably less than 2% and most preferably less than 0.5% of the triglycerides present in the oil is hydrolyzed.
   Paragraph 8. The process according to paragraph 1, wherein the diglyceride concentration is reduced by at least 30%, more preferably at least 40%, and most preferably by at least 50%.
   Paragraph 9. The process according to paragraph 1, wherein the lipase is a polypeptide having at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1.
   Paragraph 10. The process according to any of the preceding paragraphs, wherein the oil is e.g. derived from one or more of algae oil, canola oil, coconut oil, castor oil, copra oil, corn oil, distiller's corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, shea oil, tall oil, oil from halophytes, and/or animal fat, including tallow from pigs, beef and sheep, lard, chicken fat, fish oil, palm oil free fatty acid distillate, soy oil free fatty acid distillate, soap stock fatty acid material, yellow grease, used cooking oil, palm oil mill effluent and brown grease or any combination thereof.
   Paragraph 11. The process according to any of the preceding paragraphs, wherein the process is performed at temperatures in the range of 10-100° C., preferably 20-90° C.
   Paragraph 12. The process according to any of the preceding paragraphs, wherein the lipase is dosed in the range of 0.1-50000 mg enzyme protein (EP)/kg of oil.
   Paragraph 13. The process according to any of the preceding paragraphs, wherein the lipase is a liquid product, an immobilized product or as a dry powder.
   Paragraph 14. The process according to any of the preceding paragraphs, wherein the total reaction time of the process is at least 15 minutes.
   Paragraph 15. The process according to any of the preceding paragraphs, wherein the total reaction time of the process is up to 48 hours.
   Paragraph 16. The process according to any of the preceding paragraphs, wherein the amount of water added is between 0.01 and 100% (w/w) of oil.
   Paragraph 17. The process according to any of the preceding paragraphs, wherein pH is optionally adjusted during or prior to hydrolysis to optimize the reaction.
   Paragraph 18. The process according to paragraph 14, wherein the pH during hydrolysis is preferably between 3.0-7.0.
   Paragraph 19. The process according to paragraphs 14-15, wherein the pH is preferably adjusted using citric acid, phosphoric acid, sodium hydroxide and/or potassium hydroxide.
   Paragraph 20. The process according to any of the preceding paragraphs, wherein the process is performed in a batch, semi-continuous or continuous mode.
   Paragraph 21. The process according to any of the preceding paragraphs, further comprising addition of one or more additional lipases and/or phospholipases during hydrolysis.
   Paragraph 22. The process according to any of the preceding paragraphs, further comprises of presence of an organic solvent during reaction.
   Paragraph 23. The process according to paragraph 22, where the organic solvent is acetone, hexane or heptane.

### EXAMPLES

SEQ ID NO:1 of the present invention is shown as SEQ ID NO: 1 of WO2008065060.
SEQ ID NO: 2 of the present invention is shown as SEQ ID NO: 2 of WO2011067349.

### Example 1: The Surprising Effect of SEQ ID NO: 1

Fully melt the palm stearin by heating it. Weight the required amount off, add in the required weight of water and incubate the mixture to 60° C. Add the required dosage of lipase of SEQ ID NO: 1. React while mixing at 60° C. After sampling and centrifugation at 2000 g for 5 mins, take the light phase and analyze the oil for wt % FFA by titration (AOCS 5a-40 Free Fatty Acid in Crude and Refined Fats and Oils).

TABLE 1
Experimental setup

| | Trial 1 | Trial 2 | Trial 3 | Trial 4 |
|---|---|---|---|---|
| Temperature/° C. | 60 | 60 | 60 | 60 |
| g Palm Stearin | 255.59 | 151.48 | 150.74 | 150.71 |
| Dosage of SEQ ID NO: 1 | 84.8 | 84.2 | 90.8 | 91.4 |
| (mg enzyme protein/kg oil) | | | | |
| % (wt/wt) H2O | 15.0 | 49.8 | 38.0 | 44.8 |
| glycerol/g | — | — | 60.5 | 31.6 |
| g H2O | 42.51 | 148.52 | 90.91 | 120.61 |

TABLE 2
Results
wt % FFA (as palmitic acid by AOCS Ca 5a-40 method):

| | Trial 1 | Trial 2 | Trial 3 | Trial 4 |
|---|---|---|---|---|
| 0 h | 0.1 | 0.1 | 0.1 | 0.1 |
| 1 h | 5.9 | 4.8 | 2.3 | 3.7 |
| 2 h | 7.6 | 6.9 | 3.1 | 4.4 |
| 3 h | 9.0 | 8.3 | 4.8 | 5.1 |
| 4 h | 10.0 | 8.5 | — | — |
| 5 h | 11.0 | — | — | — |
| 20 h | — | — | 12.3 | 10.4 |
| 22 h | 21.0 | — | 13.1 | 10.3 |
| 23 h | — | 19.4 | — | — |
| 24 h | 22.5 | 21.4 | — | — |

FFA increases very quickly initially, which suggests that mono- and diglycerides are initially converted quickly. Then during >20 hours of reaction, a significant amount of triglyceride is converted. One would not expect 22 wt % FFA stemming from mono- and diglyceride hydrolysis alone when hydrolyzing palm stearin. Therefore, triglycerides must have been converted, especially in trials 1 and 2 above, where no glycerol was initially present. The lower conversion with glycerol present is attributed to an equilibrium between hydrolysis and esterification of the fatty acids back onto the glycerol.

These results are meant to show the common belief in the industry, that SEQ ID NO: 1 does have a significant effect on triglycerides, which is why SEQ ID NO: 1 has not been considered a viable enzyme for this application. SEQ ID NO: 1 is, for example, commonly used as enzymatic catalyst for production of triglycerides through esterification of FFA and glycerol, in the opposite reaction direction of the same hydrolysis reaction.

The inventors have found out that using SEQ ID NO: 1 normally, one would be expected to have a significant activity on triglycerides, especially when combining a sufficiently high temperature with high water dosage, long reaction time and relatively high enzyme dosage. It is thus surprising that SEQ ID NO: 1 can be made to react diglycerides with immeasurably low activity on the triglycerides.

### Example 2: Hydrolysis of Diglycerides

Fully melt the crude palm oil (CPO) by heating it up to 70° C. Weight the required oil into a 250 ml square Schott bottle. Add in the required weight of water and incubate the mixture to 50° C. or 60° C. with stirring. Add the required dosage of lipase of SEQ ID NO: 1. React in a water bath with stirrer at 60° C. with 350 rpm. Sampling in a test tube after 4 and 24 h. After sampling and centrifugation at 2000 g for 5 mins, take the light phase and analyze the oil for % FFA by titration, mono- and diglyceride content by GC and TG profile by GC (AOCS 5a-40 Free Fatty Acid in Crude and Refined Fats and Oils and AOCS Ce 5-86 Triglycerides by Gas Chromatography).

TABLE 3
Experimental setup

| Trial | T1 | T2 | T3 | T4 |
|---|---|---|---|---|
| Oil | CPO | CPO | CPO | CPO |
| CPO/g | 99 | 99 | 99 | 98 |
| Water/g | 1 | 1 | 1 | 2 |
| Dosage of SEQ ID NO: 1 | 3.4 | 34 | 3.4 | 3.4 |
| (mg enzyme protein/kg oil) | | | | |
| Temperature/° C. | 60 | 60 | 50 | 50 |
| HSM | 20,000 rpm, | 20,000 rpm, | — | — |
| | 30 sec | 30 sec | | |
| Stirring/rpm | 350 | 350 | 350 | 350 |
| Time/h | 4, 24 | 4, 24 | 4, 24 | 4, 24 |

TABLE 4
Data tabulation (wt % FFA and wt % DG)

| | T1 | T2 | T3 | T4 |
|---|---|---|---|---|
| wt % FFA (as palmitic acid by | | | | |
| AOCS Ca 5a-40 method) | | | | |
| Feed-CPO | 4.3 | 4.3 | 4.3 | 4.3 |
| 4 h | 5.9 | 7.1 | 5.1 | 6.7 |
| 24 h | 5.8 | 6.2 | 5.8 | 8.1 |
| wt % DG (by AOCS Ce 5-86 method) | | | | |
| Feed-CPO | 6.9 | 6.9 | 6.9 | 6.9 |
| 4 h | 4.4 | 3.2 | 5.3 | 3.7 |
| 24 h | 4.7 | 4.5 | 5.3 | 2.4 |

Results as shown in Table 4, T4 with higher water of 2% at 50° C. and 3.4 mg lipase/kg oil gave lowest DG content: reducing from 6.9 wt % in CPO down to 2.4 wt % after 24 h reaction. DG hydrolysis from 4 h to 24 h, 1% water seems to be too little for the system where the conversion stagnant after 4 h (both FFA and DG), perhaps the free water available at that stage (fully utilized after 4 hrs reaction) was too low to stimulate further conversion. T2 with 10x more enzyme dosage, 34 mg lipase/kg oil was able to promote faster DG reduction from 6.9 wt % to 3.2 wt % in 4 hrs. However, with low free water available, condensation occurred where the DG increased to 4.5 wt % after another 20 hrs reaction. Comparing T1 and T3 (60° C. vs. 50° C.), the difference in FFA is almost the same, but 60° C. seems to be able to achieve lower DG value (4.7 wt % vs. 5.3 wt %). Referring to Table 5, the TG profile remained unchanged after enzyme hydrolysis, showing no sign of interesterification and further substantiating the claim of little to no activity on the triglycerides.

Further, it can be concluded that the effect of SEQ ID NO: 1 in specific hydrolysis of DG 5 while not interacting with the TG in the oil at low lipase dosage. Additionally, it shows significant and impactful reduction of DG in about 4 hours.

TABLE 5
Interesterification (% of each identifiable oil component)

| | CPO | | | | |
|---|---|---|---|---|---|
| Sample | feedstock | T2-4 H | T2-24 H | T4-4 H | T4-24 H |
| % DG | 6.9 | 3.2 | 4.5 | 3.7 | 2.4 |
| C46/1-MPP | 0.7 | 0.7 | 0.7 | 0.7 | 0.7 |
| C46/2-MOM | 0.1 | 0.1 | 0.1 | 0.1 | 0.1 |
| C48/1 - PPP | 7.2 | 7.4 | 7.2 | 7.2 | 7.2 |
| C48/2 - POM | 1.6 | 1.6 | 1.6 | 1.6 | 1.6 |
| C48/3 - PLM | 0.4 | 0.4 | 0.5 | 0.5 | 0.5 |
| C50/1 - PSP | 1.3 | 1.4 | 1.3 | 1.3 | 1.3 |
| C50/2 - POP | 31.2 | 31.1 | 31.2 | 31.2 | 31.2 |
| C50/3 - MOO | 0.2 | 0.3 | 0.2 | 0.3 | 0.2 |
| C50/4 - PLP | 9.8 | 9.6 | 9.7 | 9.7 | 9.8 |
| C50/5 - MLO | 0.3 | 0.3 | 0.2 | 0.5 | 0.5 |
| C52/1 - PSS | 0.2 | 0.2 | 0.2 | 0.2 | 0.2 |
| C52/2 - POS | 5.3 | 5.4 | 5.4 | 5.3 | 5.4 |
| C52/3 - POO | 20.5 | 20.9 | 20.9 | 20.8 | 20.7 |
| C52/4 - PLS | 1.5 | 1.0 | 1.1 | 1.1 | 1.2 |
| C52/5 - POL | 8.7 | 8.7 | 8.7 | 8.7 | 8.7 |
| C52/6 - PLL | 2.0 | 2.1 | 2.0 | 2.1 | 2.1 |
| C54/1 - SSS | 0.0 | 0.0 | 0.0 | 0.0 | 0.0 |
| C54/2 - SOS | 0.6 | 0.6 | 0.6 | 0.5 | 0.6 |
| C54/3 - SOO | 2.1 | 2.1 | 2.1 | 2.1 | 2.1 |
| C54/4 - SLS | 0.2 | 0.2 | 0.2 | 0.2 | 0.1 |
| C54/5 - OOO | 2.7 | 2.7 | 2.7 | 2.7 | 2.7 |
| C54/6 - SLO | 1.0 | 1.0 | 1.0 | 1.1 | 1.0 |
| C54/7 - OLO | 1.3 | 1.3 | 1.3 | 1.3 | 1.3 |
| C54/8 - SLL | 0.2 | 0.2 | 0.2 | 0.2 | 0.2 |
| C54/9 - OLL | 0.3 | 0.3 | 0.3 | 0.3 | 0.3 |
| C54/10 - LLL | 0.0 | 0.0 | 0.0 | 0.0 | 0.0 |
| C56/1 - SOA | 0.1 | 0.1 | 0.1 | 0.1 | 0.1 |
| C56/2 - AOO | 0.0 | 0.0 | 0.0 | 0.0 | 0.0 |
| Unknown | 0.4 | 0.4 | 0.3 | 0.2 | 0.1 |
| Total (TG) | 100.0 | 100.0 | 100.0 | 100.0 | 100.0 |

### Example 3: Hydrolysis of DG Using SEQ ID NO: 2

Fully melt the crude palm oil (CPO) by heating it. Weigh the required amount off, add in 5% (wt/wt) water and incubate the mixture to 75° C. Add the required dosage of lipase of SEQ ID NO: 2. React while mixing at 75° C. After sampling, heat to 99° C. for 10 minutes and employ centrifugation at 2000 g for 5 mins, take the light phase and analyze the oil for % FFA by titration (AOCS 5a-40 Free Fatty Acid in Crude and Refined Fats and Oils) and the mono- and diglycerides by a customized HPLC method.

TABLE 6
Experimental setup

| | Trial 1 |
|---|---|
| Temperature/deg C. | 75 |
| g Crude palm oil | 30 |
| Type of enzyme | SEQ ID NO: 2 |
| mg enzyme protein/kg oil | 8.5 |
| % (wt/wt) H2O | 5 |

TABLE 7
Results
% FFA (as palmitic acid by AOCS Ca 5a-40 method), DG and
TG as normalized relative HPLC peak areas.

| | | | DG HPLC peak, | TG HPLC peak, |
|---|---|---|---|---|
| | | | normalized | normalized |
| | Time (h) | FFA (wt %) | relative area | relative area |
| CPO | 0 | 4.50 | 6.41 | 88.8 |
| Trial 1 | 24 | 8.71 | 2.39 | 87.9 |

From Table 7, it is seen that SEQ ID NO: 2 has significant and impactful reduction of DG.

### Example 4: SEQ ID NO: 1 in Combination with a PLC (Quara Boost)

Laboratory scale enzymatic water degumming was performed at 55° C. and 3 wt % total water content. Two samples of crude soybean oils with various quality were used in this experiment (Table 8). Oils were preheated to 55° C. and 30 g portions were transferred into glass tubes. Enzymes and water were added accordingly, and samples were sonicated for 5 min at 50° C. to ensure sufficient distribution and mixing of enzymes and water into oil phase. In the next step oil samples were placed in heating cabinet and incubated under gentle rotation at 55° C. for a selected time. Sequential treatment was examined. In the first step phospholipase C was added to oil samples, and after 2 h SEQ ID NO:1 was added to selected samples, thereafter both PLC and SEQ ID NO:1, were present simultaneously in reaction mixture. The enzymatic reaction was stopped after 24 h by heating oil samples to 95° C. for 10 min. In control samples only phospholipase C for 24 h or only SEQ ID NO:1 for 22 h were added. Gums and oil phase were separated by centrifugation at 600 g and 85° C. for 6 min. An upper oil phase was transferred to fresh tubes and kept for analysis. Diglycerides (DG, wt %) and Free Fatty Acids (FFA, wt %) were analyzed in oil phase. DG were analyzed by Dionex Ultimate3000 HPLC system with Corona detector, column: HypersilGold Silica 3 μm 150×4.6 mm, according to AOCS Official Method Cd 11d-96. FFA were analyzed by NaOH titration according to the AOCS Ca 5a-40 official method. Phospholipids were analyzed in crude oil samples by ³¹P NMR.

TABLE 8
Results of enzymatic degumming of soybean oil by PLC and
SEQ ID NO: 1, at 55° C. and 3% water, 24 h reaction time

| | | PLC (150 | SEQ ID NO: 1 | PLC + SEQ ID NO: 1 |
|---|---|---|---|---|
| | | ppm Quara | (6.8 ppm | (150 ppm Quara Boost product + |
| Experiment | Crude Oil | Boost | enzyme protein/ | 6.8 ppm enzyme protein/kg |
| 1 | 1 | product) | kg oil) | oil of SEQ ID NO: 1) |
| DG, wt % | 1.86 | 2.2 | 0.55 | 0.6 |
| FFA, wt % | 2.44 | 2.2 | 3.3 | 3.7 |
| Total PL, | 342 | NA* | NA | NA |
| ppm | | | | |

| | | PLC (150 | | PLC + SEQ ID NO: 1 |
|---|---|---|---|---|
| | | ppm Quara | SEQ ID NO: 1 | (150 ppm Quara Boost product + |
| Experiment | Crude Oil | Boost | (6.8 ppm E/ | 6.8 ppm enzyme protein/kg |
| 2 | 2 | product) | kg oil) | oil of SEQ ID NO: 1) |
| DG, wt % | 0.45 | 0.9 | 0.17 | 0.6 |
| FFA, wt % | 0.7 | 0.4 | 0.7 | 0.75 |
| Total PL, | 503 | NA | NA | NA |
| ppm | | | | |

*NA—not analyzed

From Table 8, it can be seen that combining SEQ ID NO: 1 with a PLC-type phospholipase yields an oil with reduced DG level relative to normal PLC degumming without the lipase of SEQ ID NO: 1. PLC-type phospholipases convert phospholipids into diglycerides and liberates the phosphate side groups. Such conversion of phospholipids is a well known type of enzymatic degumming, bringing a yield increase over traditional non-enzymatic degumming methods e.g. water/acid degumming. As a result of the PLC-catalyzed reaction, diglyceride levels in some cases increase to troublesome levels and a combination of PLC and a diglyceride active enzyme like SEQ ID NO: 1 is therefore desirable.

### Example 5: SEQ ID NO: 1 as Liquid and Immobilized Formulation

50 g of the same CPO as in example 3 above was used. 4% (wt/wt) water was added to the oil and the mixture was preheated to 50° C. Lipase was added into the mixture and reaction was run under 250 rpm stirring in a shaking incubator using 100 mL square bluecap bottles. After sampling, heat to 99° C. for 10 minutes and employ centrifugation at 2000 g for 5 mins, take the light phase and analyze the oil for % FFA by titration (AOCS 5a-40 Free Fatty Acid in Crude and Refined Fats and Oils) and the mono- and diglycerides by a customized HPLC method.

TABLE 9
Results of comparison between liquid and immobilized of SEQ ID NO: 1

| | | | TG | DG | |
|---|---|---|---|---|---|
| | | | HPLC peak, | HPLC peak, | |
| | | | normalized | normalized | |
| | | | relative area % | relative area % | wt % FFA |
| Liquid | 14.16 mg enzyme | 2 h | 88.20 | 4.24 | 6.96 |
| | protein/kg oil | 24 h | 89.27 | 2.53 | 8.06 |
| | 174.67 mg enzyme | 2 h | 87.95 | 3.61 | 8.17 |
| | protein/kg oil | 24 h | 88.48 | 2.22 | 9.08 |
| Immobilized | 14.16 mg enzyme | 2 h | 88.41 | 5.97 | 4.65 |
| | protein/kg oil | 24 h | 89.65 | 5.06 | 4.79 |
| | 174.67 mg enzyme | 2 h | 88.99 | 5.64 | 4.99 |
| | protein/kg oil | 24 h | 88.43 | 3.85 | 7.05 |

Comparing the results, the liquid formulation provides a higher rate of reaction than the immobilized formulation. The lipase of SEQ ID NO: 1 and 2 can be employed both as liquid, dry or immobilized formulation.

### Example 6: DG Hydrolysis in Presence of Solvent

60 g of CPO comprising 4.4 wt % FFA, 0.6 wt % MG, 4.9 wt % DG was used. 2 or 5% (wt/wt) water was added to the oil along with 15, 30 or 60 g of hexane. The mixture was preheated to 50° C. 0.25% (wt/wt of CPO) preparation of SEQ ID NO:1 with 0.95 wt % active enzyme protein, was added into the mixture, and reaction was performed under 500 rpm magnetic stirring in a water bath using 250 mL square bluecap bottles. After sampling, heat to 99° C. for 10 minutes. Hexane was evaporated from samples under vacuum overnight. Samples are then centrifuged at 2000 g for 5 mins, the light phase is then analyzed for % FFA by titration (AOCS 5a-40 Free Fatty Acid in Crude and Refined Fats and Oils) and the mono- and diglycerides by a customized HPLC method.

TABLE 10
Results of reaction with presence of hexane by SEQ ID NO: 1

| | | | | TG HPLC peak, | DG HPLC peak, | MG HPLC peak, | |
|---|---|---|---|---|---|---|---|
| | | | | normalized | normalized | normalized | FFA, |
| | | | | relative area % | relative area % | relative area % | wt % |
| CPO | | | | 90.1 | 4.9 | 0.6 | 4.4 |
| 25% Hexane | 2% H20 | 1 | h | 89.9 | 4.0 | 0 | 6.5 |
| | | 4 | h | 89.7 | 3.0 | 0 | 7.5 |
| | | 24 | h | 89.9 | 2.8 | 0 | 7.4 |
| | 5% H2O | 1 | h | 89.9 | 2.7 | 0 | 7.5 |
| | | 4 | h | 90.0 | 2.4 | 0 | 8.4 |
| | | 24 | h | 90.0 | 1.6 | 0 | 8.8 |
| 50% Hexane | 2% H20 | 1 | h | 90.2 | 4.0 | 0 | 5.9 |
| | | 4 | h | 89.7 | 3.8 | 0 | 7.2 |
| | | 24 | h | 90.1 | 2.5 | 0 | 7.9 |
| | 5% H2O | 1 | h | 89.6 | 3.9 | 0 | 6.8 |
| | | 4 | h | 89.9 | 2.9 | 0 | 8.3 |
| | | 24 | h | 90.8 | 1.2 | 0 | 8.9 |
| 100% Hexane | 2% H20 | 1 | h | 89.5 | 4.8 | 0 | 6.1 |
| | | 4 | h | 89.6 | 4.5 | 0 | 6.9 |
| | | 24 | h | 90.2 | 2.5 | 0 | 8.3 |
| | 5% H2O | 1 | h | 89.8 | 3.9 | 0 | 6.6 |
| | | 4 | h | 90.0 | 2.9 | 0 | 8.4 |
| | | 24 | h | 90.4 | 1.8 | 0 | 9.2 |

DG hydrolysis with hexane/oil mixture coming directly from the extraction step before hexane is removed and the oil is isolated. Results above indicate that 50% hexane is preferable to 25% and 100%, because DAG can be hydrolyzed the most.

While there has been shown and described what are at present considered the preferred embodiments of the invention, it will be obvious to those skilled in the art that various changes and modifications can be made therein without departing from the scope of the invention defined by the appended claims.

1-15. (canceled)

16. A process for reducing and/or removing diglyceride content without substantial interesterification of triglycerides in an oil, said process comprising steps of:
a. providing an oil or fat; and
b. hydrolysis of diglycerides in said oil with water in the presence of a lipase having at least 80% sequence identity to C. antarctica lipase B (CALB).

17. The process according to claim 16, further comprises the step of separation of a light phase and a heavy phase after hydrolysis.

18. The process according to claim 17, wherein the light phase comprises the oil with reduced diglyceride and increased FFA.

19. The process according to claim 17, wherein the heavy phase comprises water, lipase, and glycerol.

20. The process according to claim 17, wherein the heavy phase is partially or fully recycled into hydrolysis step.

21. The process according to claim 17, wherein free fatty acid is separated from the oil present in the light phase.

22. The process according to claim 16, wherein less than 10% of the triglycerides present in the oil is hydrolyzed.

23. The process according to claim 16, wherein the diglyceride concentration is reduced by at least 30%.

24. The process according to claim 16, wherein the lipase is C. antarctica lipase B (CALB).

25. The process according to claim 16, wherein the oil is derived from algae oil, canola oil, coconut oil, castor oil, copra oil, corn oil, distiller's corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, shea oil, tall oil, oil from halophytes, animal fat, palm oil free fatty acid distillate, soy oil free fatty acid distillate, soap stock fatty acid material, yellow grease, used cooking oil, palm oil mill effluent and brown grease, or any combination thereof.

26. The process according to claim 16, wherein the amount of water added is between 0.01 and 100% (w/w) of oil.

27. The process according to claim 16, wherein pH is adjusted during or prior to hydrolysis to optimize the reaction.

28. The process according to claim 16, further comprising addition of one or more additional lipases and/or phospholipases during hydrolysis.

29. The process according to claim 16, further comprising an organic solvent during reaction.

30. The process according to claim 29, where the organic solvent is acetone, hexane or heptane.
